(19) United States
(12) Patent Application Publication
(10) Pub. No.: US 2010/0087898 A1
(43) Pub. Date: Apr. 8, 2010

(54) APPARATUS AND METHOD FOR DELIVERING LIGHT THERAPY

(75) Inventors: Robert Marc Clement, Swansea (GB); Michael Noel Kiernan, Swansea (GB); Gareth Ian Stockman, Swansea (GB)

Correspondence Address: KOHN & ASSOCIATES, PLLC, 30500 NORTHWESTERN HWY, SUITE 410, FARMINGTON HILLS, MI, 48334, US

(73) Assignee: CYDEN LTD., Swansea (GB)

(21) Appl. No.: 12/445,538

(22) PCT Filed: Oct. 15, 2007
(86) PCT No.: PCT/GB07/03985

§ 371 Date: Nov. 11, 2009

(30) Foreign Application Priority Data
Oct. 14, 2006 (GB) 0620436.6
Jun. 21, 2007 (GB) 0711999.3

(51) Int. Cl. A61N 5/06

(52) U.S. Cl. 607/88

(57) ABSTRACT

The apparatus comprises a removable coating formulation for a surface of a vertebrate animal body (such as an aqueous coating formulation); with at least two light-emitting diodes which can be secured to the surface by the formulation such that illumination therefrom can be directed towards tissue in the animal body. The light-emitting diodes are such that, when connected to an electrical power supply, they have an illumination power output substantially in the range causing photochemical effects in the animal tissue, and they are provided with a connection to an electrical power supply.

The present invention relates to apparatus and method for delivering light therapy to a subject, for example, for use in stimulation of cartilage and other collagen-containing structures to encourage or promote regrowth thereof, as well as a method of attaching a system for delivering light therapy to a subject.

The treatment of patients by light irradiation is a well established technique, typically for photodynamic therapy (when the light is used together with a pharmaceutical), for therapeutic treatment of skin or the like, when light alone is used to treat the skin, or for cosmetic treatment of skin or the like.

A traditional delivery system for applying light irradiation to a subject has been a stand-alone light source, such a laser source or an intense pulsed light source. Such a system is cumbersome and is frequently only suitable for use in hospitals, clinics or the like where the subject visits for treatment.

There have recently been developed systems which permit a light source or a plurality of light sources to be attached to the body being treated.

For example, WO 2006/081221 discloses a phototherapy device including an array of LEDs attached to a carrier in the form of a glove, sock, patch, cap, wrap etc. US2006/0271131 concerns phototherapy apparatus for cartilage regeneration, the apparatus having a support to conform to the shape of a body part and a number of radiation sources (such as laser diodes) secured to the support. U.S. Pat. No. 6,096,606 discloses and claims a light therapy patch which comprises an array of light sources mounted on a flexible patch. WO 2007/047892 discloses an externally wearable light therapy treatment system with a wearable device (such as a knee, wrist, back, ankle or elbow brace, or a patch, glove, sock or cap) to which is mounted a series of light sources such as an array of LEDs. Other similar disclosures are in WO 2007/002073, WO 2006/081221, US2005/0237739 and US2004/0220513.

All of the systems described above have the light sources in pre-assigned positions on a backing or carrier member. They are then secured or attached to the body either by means of an adhesive, or by means of a part which wraps or fits around the body or body part. While such arrangements are useful for a number of purposes, greater flexibility in ways of attachment of the light sources to the body would be desirable.

According to a first aspect of the invention, therefore, there is provided apparatus (typically in the form of a kit) for delivering light therapy, which apparatus comprises:
a) a spreadable coating formulation for forming a removable translucent or transparent coating layer on a surface of a vertebrate animal body;
b) a plurality of light-emitting diodes (LEDs) which can be secured to the surface by means of the formulation such that illumination therefrom can be directed towards tissue in the animal body, the LEDs having an illumination power output substantially in the range causing photochemical effects in tissue of the animal body; and
c) means for connection of the LEDs to an electrical power supply to permit the LEDs to illuminate the tissue and cause photochemical effects therein.

The present invention further comprises a method of attaching a system for delivering light therapy to an animal subject, which method comprises
(i) providing a plurality of light-emitting diodes (LEDs) having an illumination power output substantially in the range causing photochemical effects in tissue of a vertebrate animal body;
(ii) securing the LEDs to the surface by means of a spreadable coating formulation so as to form a removable translucent or transparent coating layer on a surface of the subject with optical output from the LEDs directed towards the surface; and
(iii) providing connection of the LEDs to an electrical power supply to permit illumination of the LEDs by the power supply.

The LEDs therefore become embedded or incorporated in a layer of the coating formulation, with at least part of the thickness of the layer separating the LEDs from the surface and permitting transmission of optical output from the LEDs to the surface.

The present invention further provides a method of delivering light to a vertebrate animal subject (for cosmetic or therapeutic purposes), which method comprises illuminating tissue of the vertebrate animal by means of an array of LEDs having a power output substantially in the range causing photochemical effects in the animal tissue, in which the LEDs are secured to a surface of the animal by a removable coating formulation as described above.

In one embodiment of the invention, the coating formulation may comprise a flexible material which permits the coating formulation to form a carrier member which can conform to a body part, for example, by permitting the LEDs to be wrapped around the body part to be treated and secured thereto by the flexible material.

Examples of suitable such flexible materials include plastics coating film-forming polymers of, for example, a settable or curable gel, or a thixotropic coating formulation, which is preferably substantially free of irritant materials.

The coating formulation is preferably one which is translucent or transparent to at least part of the visible spectrum, namely that part of the visible spectrum corresponding to the visible light emitted by the LEDs, such as in the green and/or yellow part of the visible spectrum. It is particularly preferred that the coating formulation is transparent over at least the majority of the visible spectrum.

The coating formulation preferably contains a film-forming polymer in a predominantly aqueous carrier (which is preferably substantially free of organic solvents such as alcohols in order to minimise painful stinging). Examples of suitable film-forming polymers include water-soluble biologically harmless polymers such as polyvinyl alcohol, alginates, carrageenan, cellulose or cellulose derivatives, or biologically harmless gel-forming polymers such as silicones.

Although the formulation should be substantially free of deleterious ingredients, it may contain beneficial ingredients such as bactericides, antiseptics, antibiotics or other pharmaceutically active ingredients.

As indicated, the LEDs employed in the apparatus according to the invention are such that they result in an illumination power output which is such as to induce substantially only photochemical effects, substantially without thermal effects (unlike the thermal effects described in, for example, WO 00/74782). Light irradiation capable of inducing only photochemical effects generally has a power density in watts/cm² of less than 1000, especially less than 100. The LEDs may be arranged to be pulsed if provided with a suitable controller; alternatively, the apparatus may include timer means to control the time of illumination of the body tissue, and/or intervals between successive pulses of illumination.

Such illumination may be suitable for stimulating collagen regrowth in collagen-containing structures. Photochemical effects achieved using apparatus or kits according to the invention have been shown in vitro in a study on 18 month old bovine cartilage (known to be equivalent to typical human cartilage of a 30 to 50 year old person) to stimulate collagen regrowth and in particular chondrocyte activity, without significant cell necrosis or other adverse effects.

The present invention therefore further provides a method of stimulating collagen regrowth in collagen-containing structures (for example, stimulating collagen regrowth mediated by chondrocyte activity, or collagen regrowth in tendons or ligaments), which method comprises illuminating body tissue containing cartilage by means of an array of LEDs having a power output substantially in the range causing photochemical effects in the body tissue, the LEDs being secured to a surface of a vertebrate animal by a removable coating formulation as described above.

The formulation is typically applied to the surface of the body being treated by spreading; when it is applied by spreading it may be spread by any suitable spreader, such as a spatula, roller or brush. The apparatus or kit therefore preferably includes means for applying the formulation by spreading, typically in metered amounts so as to provide coverage for a predefined area of the surface.

The apparatus according to the invention may include a template of predetermined shape and size, so that the coating formulation can be applied to the surface so as to cover a predetermined area and shape of the surface, such as a strip, rectangle or the like. The method according to the invention therefore may include applying the coating formulation to the surface to cover a predetermined shape and area, using such a template to define the shape and area to which the coating formulation is applied.

The LEDs used in the apparatus and method according to the invention may, in some embodiments, provide a selected wavelength of illumination. It is particularly preferred that they should provide a wavelength of illumination of less than 600 nanometres, such as 540 to 560 nanometres (green) or 570 to 590 nanometres (yellow) both of which are wavelengths at which haemoglobin has a characteristic absorption peak. Haemoglobin is, of course, an active ingredient of blood, and when the collagen structure is vascular (for example, in ligament or tendon), absorption by haemoglobin present in the blood helps to impart energy to the collagen structure; when the collagen structure is avascular, the energy absorption by haemoglobin is believed to be transferred to synovial fluid, which is in contact with an outer layer of a collagen structure such as cartilage. This is believed to stimulate the regeneration of cartilage by apotosis (that is, from an outer surface inwards).

It is further preferred that the apparatus and method according to the invention are arranged to provide illumination for at least one predetermined time period (for example, by use of a timer mechanism or the like). In some embodiments of the invention, such a timer mechanism may be arranged to provide sequential doses of illumination from the LEDs as pulses or at spaced intervals of time (which may be pre-selected, or chosen by the user or health care professional or the like).

In some embodiments of the invention, the apparatus may further include pre-programmed control means which are arranged to trigger the illumination of the LEDs at predetermined time intervals for predetermined doses.

The LEDs may be arranged on the body surface as an array, such as a single row, a plurality of rows, or a suitable geometric shape. The LEDs are preferably mounted so as to directly face and illuminate the surface of the body part to which they are to be attached, for extracorporeal illumination thereof. If there is any intervening material between the LEDs and the surface of the body, it should be substantially transparent to the wavelength or wavelengths of the light which causes the desired photochemical effects. The LEDs may be mounted to a base in such a way that one or more of the LEDs may be replaced, for example if one fails or if is wished to change the wavelength, power output or other parameters of the illumination.

The power supply is typically provided by, for example, battery or a mains supplied transformer. The kit may include a circuit board to which the LEDs can be mounted, together with suitable conductors for connection to the power supply. In another embodiment, a pair of conductive paths, such as printed metal tracks, may be provided on the obverse of the LEDs (a face remote from that intended to be directed towards the user's body). A patch or the like with a pair of spaced electrodes may be positioned so that the spaced electrodes contact such paths, thereby permitting power supply to the LEDs.

In some aspects of the invention, the apparatus may include further therapeutic elements—for example, it may contain a heating element, a cooling element or the like. When a heating element is provided, it may for example be electrically heated or a pre-heated module (for example of thermal storage material). When a cooling element is provided it may for example be a Peltier cooler or a pre-cooled module (for example, of cooled gel material or the like).

Preferred features of the apparatus and method according to the invention will now be further described, with reference to specific embodiments given by way of example only, in which:

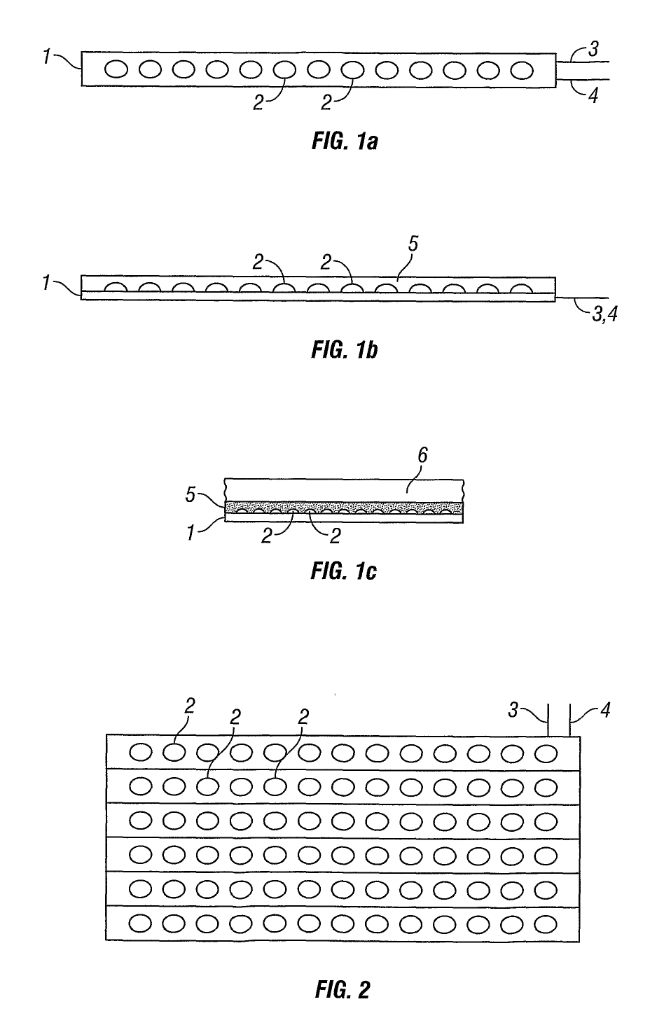

FIGS. 1a and 1b are respective schematic plan and side views of a first embodiment of an array of LEDs applied and attached to a skin surface in a method according to the invention, and FIG. 1c is a sectional view of the array of FIGS. 1a and 1b when attached to the skin surface;

FIG. 2 is a schematic plan view of a second embodiment of an array of LEDs applied and attached to a skin surface in a method according to the invention;

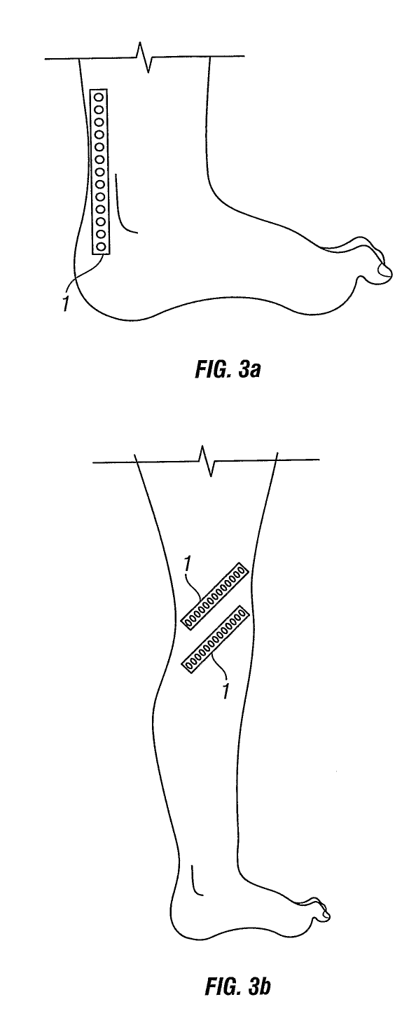

FIGS. 3a and 3b are schematic representations showing the array of FIGS. 1a and 1b when applied respectively to the ankle region (FIG. 3a) and to the knee (using two such arrays in FIG. 3b).

Referring to the drawings, and initially to FIGS. 1a and 1b, there is shown a longitudinally extending row of LEDs 2 secured to a body surface 6 (see FIG. 1c) by a coating layer 1 in which the LEDS are embedded, with part 5 of the coating layer 1 between the LEDs and the body surface. The row of LEDs 2 permits illumination of a body part when bonded thereto by coating layer 1. There are thirteen such LEDs shown in the illustrated embodiment, but this number is not critical and any appropriate number may be used.

At one end of the row of LEDs 5 is a pair of electrical connectors 3,4 arranged to be connected to a power supply (such as a battery, mains powered transformer or the like) by means of which the entire row of LEDs may be illuminated simultaneously.

Referring to FIG. 1c, the LEDs 5 are secured to the body surface 6 of a user by the removable adhesive coating layer 1. The LEDs may be secured to any suitable body part, such as a wrist, knee, elbow or chest, by adhesion.

Referring now to FIG. 2, there is shown a similar arrangement to that illustrated in

FIGS. 1a and 1b (in which like parts to those in FIGS. 1a and 1b are denoted by like reference numerals), except that the LEDs are arranged on the surface of the relevant body part in an array comprising six rows each containing thirteen LEDs. Referring now to FIG. 3a, there is shown a strip 1 similar to that of FIGS. 1a and 1b; secured to a user's ankle adjacent the Achilles heel (with the LEDs facing the skin) using a coating formulation according to the invention.

Referring now to FIG. 3b, there is shown a pair of strips 1 similar to that of FIGS. 1a and 1b secured to a user's knee (with the LEDs facing the skin) by a suitable coating formulation.

1. Apparatus for delivering light therapy, which apparatus comprises:
a) a spreadable coating formulation for forming a removable translucent or transparent coating layer on a surface overlying tissue of a vertebral animal body;
b) a plurality of light-emitting diodes (LEDs) which are adapted to be secured to said surface by means of said formulation such that illumination from said LEDs can be directed towards tissue, said LEDs being rated to produce an illumination power output substantially in the range causing photochemical effects in tissue of said animal body; and
c) connection means for connection of said LEDs to an electrical power supply to permit said LEDs to illuminate said tissue and cause photochemical effects in said tissue.

2. Apparatus according to claim 1, wherein the coating formulation is such as to form a flexible material adapted to conform to a body party overlying said tissue.

3. Apparatus according to claim 1, wherein the illumination power output has a maximum which is such as to induce substantially only photochemical effects in living tissue.

4. Apparatus according to claim 1, wherein light emitting diodes emit illumination predominantly in the range 540 to 560 nanometers or in the range 570 to 590 nanometers.

5. Apparatus according to claim 1, which further includes pulse means for pulsing the illumination output of said light emitting diodes.

6. Apparatus according to claim 1, including timer means for controlling at least one of the time duration of said illumination output and time intervals between successive doses of said illumination output.

7. Apparatus according to claims 1, including pre-programmed control means arranged to trigger the illumination at predetermined time intervals for predetermined doses of said illumination.

8. Apparatus according to claim 1, which includes a power supply for said light emitting diodes.

9. Apparatus according to claim 1, wherein the coating formulation is aqueous.

10. Apparatus according to claims 1, which includes means for applying the coating formulation by spreading.

11. A method of attaching a system for delivering light therapy to a subject, which method comprises: providing a plurality of light-emitting diodes (LEDs) having an illumination power output substantially in the range causing photochemical effects in tissue of a vertebrate animal body; applying a spreadable coating formulation to said surface together with said LEDs so as to form a removable translucent or transparent coating layer on the surface which layer secures said LEDs to said surface, the LEDs being arranged such that optical output from the LEDs is directed in use towards the surface; and providing connection of the LEDs to an electrical power supply to permit illumination of the LEDs by the power supply.

12. A method according to claim 11, wherein the coating formulation is applied to conform to a part of the animal body.

13. A method according to claim 11, wherein the coating formulation is aqueous.

14. A method according to claims 11, in which the coating formulation by applied by spreading.

15. A method according to claims 11, in which there is employed apparatus according to claim 3.

16. A method of delivering light to a vertebrate animal tissue, which method comprises illumination the tissue by means of an array of LEDs having a power output substantially in the range causing photochemical effects in the animal tissue, in which said array of LEDs is secured to a surface of the animal by a removable coating layer which has been applied to said surface by spreading a translucent or transparent coating formulation to said surface.

17. A method of stimulation collagen regrowth in collagen-containing structures in body tissue containing cartilage, which method comprises illuminating said body tissue by means of an array of LEDs having a power output substantially in the range causing photochemical effects in said body tissue, said array of LEDs being secured to a surface of a vertebrate animal by a removable coating layer which has been applied to said surface by spreading a translucent or transparent coating formulation to said surface.

18. A method according to claim 17, wherein said coating formulation is aqueous.
